(19) United States
(12) Plant Patent Application Publication
(10) Pub. No.: US 2009/0106870 P1
(43) Pub. Date: Apr. 23, 2009

(54) Hybrid tea rose plant named 'MEIZOELE'

(75) Inventor: Alain A. Meilland, Antibes (FR)

Correspondence Address: BUCHANAN, INGERSOLL & ROONEY PC, POST OFFICE BOX 1404, ALEXANDRIA, VA, 22313-1404, US

(73) Assignee: CP DELAWARE, INC., West Grove, PA (US)

(21) Appl. No.: 12/314,474

(22) Filed: Dec. 11, 2008

Related U.S. Application Data
(63) Continuation of application No. 11/502,459, filed on Aug. 11, 2006.
Application No. 11/502,459 is a continuation of application No. 10/832,331, filed on Apr. 27, 2004, now abandoned.
Application No. 10/832,331 is a continuation of application No. 10/288,426, filed on Nov. 6, 2002, now abandoned.
Application No. 10/288,426 is a continuation of application No. 09/499,673, filed on Feb. 8, 2000, now abandoned.

(51) Int. Cl. A01H 5/00

(52) U.S. Cl. PLT/131

(57) ABSTRACT

A new and distinct variety of Hybrid Tea rose plant is provided which abundantly forms attractive double bicolored blossoms that are yellow bordered with red in coloration. The plant exhibits a vigorous growth habit, semi-glossy dark green foliage, and very good disease resistance. The dark green foliage contrasts nicely with the lighter colored blossoms. The new variety is particularly well suited for growing as attractive ornamentation in the landscape such as in parks and gardens.

This is a Continuation of U.S. Plant patent application Ser. No. 11/502,459, filed Aug. 11, 2006, which is a Continuation of U.S. Plant patent application Ser. No. 10/832,331, filed Apr. 27, 2004 (now abandoned), which is a Continuation of U.S. Plant patent application Ser. No. 10/288,426, filed Nov. 6, 2002 (now abandoned), which is a Continuation of U.S. Plant patent application Ser. No. 09/499,673, filed Feb. 8, 2000 (now abandoned).

### BOTANICAL/COMMERCIAL CLASSIFICATION

Rosa hybrida/Hybrid Tea Rose Plant

### VARIETAL DENOMINATION

cv. Meizoele

### SUMMARY OF THE INVENTION

The new variety of Hybrid Tea rose plant was created by artificial pollination wherein two parents were crossed which previously had been studied in the hope that they would contribute the desired characteristics. The female parent (i.e., the seed parent) of the new variety was the ‘Meinarval’ variety (non-patented in the United States). The male parent (i.e., the pollen parent) was the ‘Lapdal’ variety (non-patented in the United States). The parentage of the new variety can be summarized as follows:

‘Meinarval×Lapdal.’

The seeds resulting from the above pollination were sown and small plants were obtained which were physically and biologically different from each other. Selective study resulted in the identification of a single plant of the new variety.

It was found that the new variety of Hybrid Tea rose plant of the present invention possesses the following combination of characteristics:

(a) exhibits a vigorous growth habit,

(b) abundantly forms attractive double bicolored blossoms that are yellow bordered with red in coloration,

(c) forms attractive dark green semi-glossy foliage that contrasts well with the blossoms, and

(d) is particularly well suited for growing as attractive ornamentation in the landscape.

The disease resistance of the new variety is very good.

The new variety of the present invention can be readily distinguished from its parental varieties. The ‘Meinarval’ female parent forms very elongated high-pointed buds that are distinct from the shorter and globular buds of the new variety. The ‘Lapdal’ male parent forms dissimilar cream blossoms that are edged with carmine.

The new variety of the present invention also can be readily distinguished from the ‘Meinomad’ (U.S. Plant Pat. No. 8,248) and ‘Meibosnio’ (U.S. Plant Pat. No. 12,876) rose varieties. More specifically, the ‘Meinomad’ variety forms flowers having a substantially larger diameter of approximately 14 cm on average, and the ‘Meibosnio’ variety forms flowers having substantially more petals that commonly number approximately 70 to 75 flower on average.

The new variety well meets the needs of the horticultural industry and can be grown to advantage in parks and gardens.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meizoele’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during October while budded on Rosa froebelli understock and growing in outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface; and

FIG. 15—illustrates a specimen of a leaf with five leaflets—plan view—under surface.

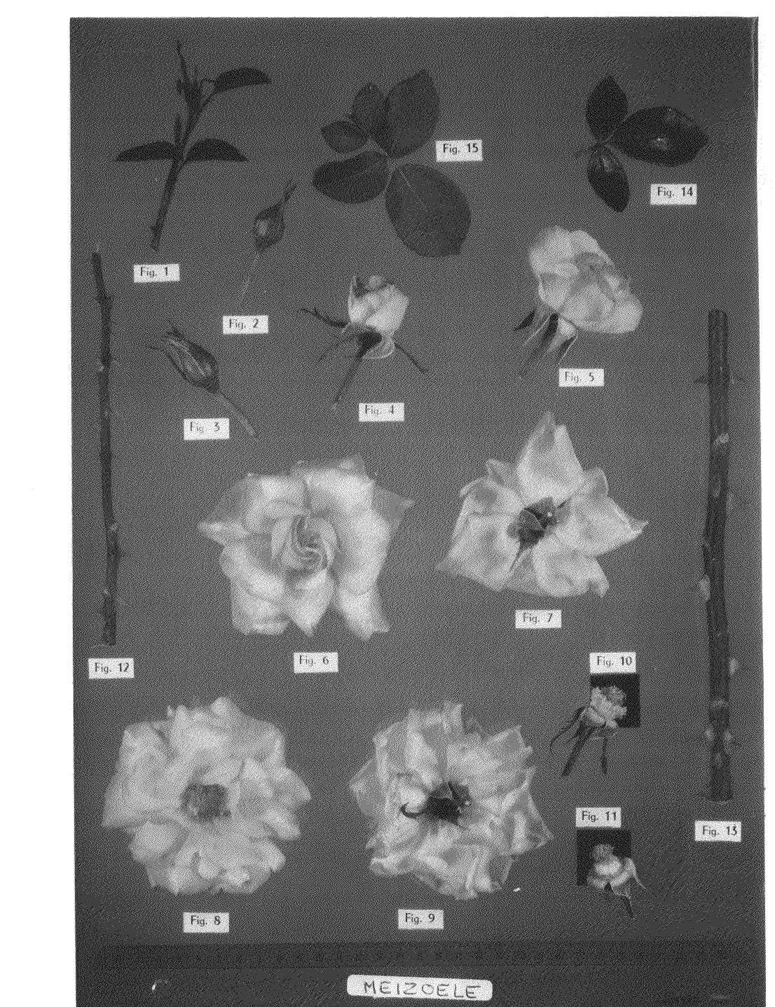

### DETAILED DESCRIPTION

The chart used in the identification of the colors is that of The Royal Horticultural Society (R.H.S. Colour Chart). The description is based on the observation of two year-old plants during October while budded on Rosa froebelli understock and growing outdoors at Le Cannet des Maures, Var, France. The coloration in common terms precedes reference to the chart in some instances. Such terminology is to be accorded its ordinary dictionary significance.
Class: Hybrid Tea.
Plant:
   Height.—approximately 80 to 90 cm at the end of the second growing season. The width is approximately 75 to 85 cm at the end of the growing season.
   Habit.—semi-erect.
Branches:
   Color.—young stems: smooth in texture, Yellow-Green Group 146A with some anthocyanin coloration near Greyed-Purple Group 183C. adult wood: smooth in texture, lighter green, Yellow-Green Group 146C.
   Thorns.—size: large (as illustrated), and commonly average approximately 1.5 cm in length. quantity: medium (as illustrated). color: Greyed-Orange Group 163A with some anthocyanin coloration near Greyed-Red Group 178C on young stems and Havana brown, Greyed-Orange Group 165B, on adult wood.
Leaves:
   Size.—a five-leaflet leaf commonly is approximately 6.5 cm in length on average, and approximately 4.5 cm in width on average.
   Stipules.—adnate, pectinate, and rather broad. The color is near Yellow-Green Group 148A, the length is approximately 1.8 to 2.1 mm the width is approximately 6 to 9 mm, and the upper and lower surfaces are smooth. There is some anthocyanin coloration near Greyed-Purple Group 183C when young.
   Petioles.—upper surface: near Yellow-Green Group 148A with slight anthocyanin coloration of Greyed-Red Group 178A and non-glandular. under surface: commonly free of prickles. length: approximately 2 to 4 cm on average from the point of attachment to the first leaflets.
   Rachis.13 surface texture: smooth. color: near Yellow-Green Group 148A on the upper surface and near Yellow Green Group 146B on the under surface.
   Leaflets.—number: 3, 5 (most often), and 7. shape: elliptic with an acute apex and an obtuse base. serration: single and regular (as illustrated). texture: rather firm, semi-glossy on the upper surface and dull on the under surface. general appearance: dark green. color (young foliage): upper surface: Green Group 138A with anthocyanin coloration near Greyed-Purple Group 183A. under surface: lighter green, Green Group 138B. color (adult foliage): upper surface: dark green, Green Group 139A. under surface: Yellow-Green Group 147A.
Inflorescence:
   Number of flowers.—usually one flower per stem.
   Peduncle.—rigid, medium in thickness, Yellow-Green Group 147C with slight anthocyanin coloration near Greyed-Red Group 178C when young, bear a few prickles, and the length is approximately 6 cm on average.
   Sepals.—upper surface: tomentose and greenish, Yellow-Green Group 148C, with some reddish coloration. under surface: Yellow-Green Group 146B, and commonly with some extensions (as illustrated). shape: elongated and pointed. size: approximately 2.5 to 3.2 cm in length.
   Buds.—shape: globular. size: large. length: approximately 3 cm on average. width: approximately 1.5 cm on average. color upon opening: upper surface: near Yellow-Orange Group 14A to 14C suffused with Red Group 46A to 46C. under surface: near Yellow-Orange Group 14B to 14C suffused with Red Group 46A to 46C.
   Flower.—shape: cup-shaped. diameter: approximately 11 to 12 cm on average. color (when opening begins): upper surface: near Yellow-Orange Group 14A to 14C suffused with Red Group 46A to 46C. under surface: near Yellow-Orange Group 14B to 14C suffused with Red Group 46A to 46C. color (when blooming): upper surface: near Yellow Group 12A to 12D bordered with Red Group 47C. under surface: near Yellow Group 12B to 12D bordered with Red Group 47C. color (at end of opening): upper surface: near Yellow Group 12A to 12D bordered with Red Group 47C. under surface: near Yellow Group 12B to 12D bordered with Red Group 47C. lasting quality: very long and approximately 8 to 10 days on the plant. The lasting quality in a vase has not been determined since this variety is primarily intended for ornamentation outdoors. petal number: commonly approximately 35 to 40 on average. petaloids: approximately 10 on average irregularly shaped petaloids sometimes are displayed at the center of the blooms, and are substantially the same coloration as the petals. petal shape: wedge-shaped and oblong, the margins are reflexed (as illustrated), the length is approximately 4 to 5 cm, the width is approximately 2.5 to 4 cm, and the surface is smooth. The petal apex is mucronate. Such apex configuration is most prominent as the blossoms first open, and becomes less noticeable as the blossoms age. On a newly open blossom the base is truncate to rounded to broadly cuneate, and changes to broadly cuneate to narrowly cuneate in mature blossoms. petal arrangement: verticillate. petal drop: good, the petals commonly detach cleanly. fragrance: none. stamen number: approximately 109 on average. anthers: near Yellow-Orange Group 15C in coloration, and approximately 2 to 3 mm in length. filaments: near Yellow-Orange Group 15C in coloration, and approximately 1 to 5 mm in length. pistils: approximately 95 on average. stigmas: near Yellow Group 8D in coloration. styles: the coloration is near White Group 155D and near Red Group 40D approaching the stigma, and the length is approximately 4 to 9 mm. pollen: near Yellow-Orange Group 21A. receptacle: near Yellow-Green Group 146B in coloration, possesses a smooth surface texture, funnel-shaped in longitudinal section, and approximately 1 cm in length.
Development:
   Vegetation.—vigorous.
   Blooming.—very abundant.
   Resistance to diseases.—very good with respect to Marsonina.
   Resistance to frost.—very good. Has withstood −10° C. without damage to mature wood.
   Ability to form hips.—none observed in open fields.
   Resistance/susceptibility to pests.—no special resistance/susceptibility observed.

I claim:

1 . A new and distinct variety of Hybrid Tea rose plant characterized by the following combination of characteristics:
(a) exhibits a vigorous growth habit,
(b) abundantly forms attractive double bicolored blossoms that are yellow bordered with red in coloration,
(c) forms attractive dark green semi-glossy foliage that contrasts well with the blossoms, and
(d) is particularly well suited for growing as attractive ornamentation in the landscape;
substantially as herein shown and described.
